(19) United States
(12) Patent Application Publication
(10) Pub. No.: US 2008/0124780 A1
(43) Pub. Date: May 29, 2008

(54) Hybrid immobilized catalytic system with controlled permeability

(75) Inventors: Mansoor M. Amiji, Attleboro, MA (US); Ehab S. Taqieddin, Boston, MA (US)

Correspondence Address: WEINGARTEN, SCHURGIN, GAGNEBIN & LEBOVICI LLP, TEN POST OFFICE SQUARE, BOSTON, MA, 02109, US

(73) Assignee: NORTHEASTERN UNIVERSITY

(21) Appl. No.: 12/011,126

(22) Filed: Jan. 24, 2008

Related U.S. Application Data
(62) Division of application No. 10/501,130, filed on Jul. 12, 2004.
(60) Provisional application No. 60/347,234, filed on Jan. 10, 2002.

(51) Int. Cl. C12N 11/04; C07K 16/00; C07H 21/04

(52) U.S. Cl. 435/182; 530/387.1; 536/23.1

(57) ABSTRACT

An immobilized catalytic system comprising a carrier layer containing a catalytic entity and a permeable screening layer for providing controlled access between the immobilizing catalytic entity and the surrounding environment and methods of making such systems are disclosed. The carrier layer includes the catalytic entity mixed with a neutral or anionic carrier polymer, which may or may not be cross-linked with a cross-linking agent. The screening layer over the carrier layer includes a matrix of a cationic polymer that is permeable to molecules processed by, produced by or acted upon by the catalytic entity but is not permeable to the catalytic entity itself. Any counter ion to the neutral or anionic carrier polymer cannot be the same as the cationic polymer of the screening layer, and any counter ion to the cationic polymer cannot be the same as the neutral or anionic carrier polymer.

### CROSS REFERENCE TO RELATED APPLICATIONS

This application is a divisional of U.S. patent application Ser. No. 10/501,130 filed on Jul. 12, 2004 entitled, HYBRID IMMOBILIZED CATALYTIC SYSTEM WITH CONTROLLED PERMEABILITY, which is a 35 U.S.C. 371 filing of International Application No. PCT/US03/00738 filed Jan. 10, 2003, entitled HYBRID IMMOBILIZED CATALYTIC SYSTEM WITH CONTROLLED PERMEABILITY, which claims the priority of U.S. Provisional Application No. 60/347,234 filed Jan. 10, 2002 entitled, PERMSELECTIVE HYBRID MICROCAPSULES FOR IMMOBILIZATION TECHNOLOGY, the whole of which are hereby incorporated by reference herein.

### STATEMENT REGARDING FEDERALLY SPONSORED RESEARCH OR DEVELOPMENT

N/A

### BACKGROUND OF THE INVENTION

Methods of delivery of proteins or whole cells for commercial or therapeutic purposes have garnered a great deal of attention in the last few decades. Recently, attention has been focused on the possibility of using immobilized enzymes or whole cells as useful commercial or therapeutic system.

Enzymes are biological catalysts, responsible for the countless reactions in our bodies as well as in our surrounding environment. The basic idea behind enzyme immobilization is to entrap the protein in a semi-permeable support material, which prevents the enzyme from leaving while allowing substrates, products and co-factors, to pass through. When an immobilized enzyme is used in vivo, the support material can prevent immune rejection of the enzyme by halting antibody recognition and other rejection processes. This concept has also been extended to living cells, where a living cell is immobilized in a matrix. A general model of an immobilized enzyme system according to the prior art is shown in FIG. 1.

Cells have been immobilized with good in vitro success. Clinical tests on immobilized cells are still very recent, and the types of cells immobilized have been restricted to their ability to secrete therapeutic proteins. Cells have also been immobilized for industrial purposes. For example, Lactobacillus casei was immobilized in alginate microcapsules for lactic acid production from broth media (Yoo et al., 1996). Cells immobilized in liquid core capsules show better efficiency compared to cells in a capsule with a solid core due to higher growth rates (Yoo et al., 1996). A liquid core can be made in several ways. In one method, alginate is cross-linked in a diluted solution of calcium chloride. Another method is to cross-link alginate with calcium chloride, followed by an additional cross-linking step with poly (L-lysine), and then to expose the capsule to a calcium chelating solution. An additional method for liquid core capsule formation has been proposed in which a chitosan solution is dropped into an alginate solution, resulting in a chitosan liquid core droplet with a solid alginate coat (Daly et al., 1988). The coat was further solidified by cross-linking with calcium chloride.

Enzymes have been immobilized by several methods either through covalent or non-covalent means. For covalent immobilization, an enzyme is attached to the supporting material by the formation of a covalent bond. Covalent immobilization provides for a long-term retention of the enzyme on the support structure. This type of immobilization requires some knowledge about the enzyme composition and structure, however, and the reactive group on the enzyme being attached must be selected from sites other than the active site in order for the enzyme to retain activity.

Non-covalent immobilization is a popular technique for immobilizing an enzyme. The elimination of chemical reactions, organic contaminants and purification steps makes it an easy and desirable method for immobilization. Among the non-covalent methods used for immobilization is carrier binding (Gemeiner, P., 1992). This term includes ionic binding of the enzyme to the support material, physical adsorption or metal binding. This type of immobilization is generally reversible, and the enzyme can be displaced from the binding site.

Another method for non-covalent immobilization is enzyme entrapment. This method includes immobilization in cross-linked fibers, liposomes, microcapsules or hollow fibers. Enzyme entrapment is usually preferred due to its simplicity and practicality on large scale. Entrapment has also been used for cells and DNA as well as for enzymes. Immobilization techniques such as covalent binding, matrix entrapment and encapsulation are described in Taylor et al., 1991.

Many factors play a role in the immobilization process, and certain basic criteria should be observed in order to achieve maximum benefit from the immobilized system. The immobilized system should retain the bioactivity of the enzyme or cellular structure it contains. The system should be biocompatible with living tissue if intended for in vivo applications. Generally, non-biodegradable systems are required. Finally, large scale reproducibility at low cost is required for any economically viable system.

Many different polymers have been used to immobilize enzymes, organelles and cells (Taylor et al., 1991; Gemeiner, P., 1992). Because of the many requirements needed to form a suitable carrier for enzymes, no polymer yet has been classified as the ideal polymer for immobilization. Polymers of natural, synthetic or semi-synthetic origin have been tested. The polymer of choice should be non-biodegradable. The polymer should also be biocompatible, as many of those immobilized systems are intended for in vivo applications. In some cases, where the immobilized system is intended for in vitro or any other application, this requirement may be reduced. The support material should also have no degrading effect on the enzyme. The final immobilized system should have enough mechanical strength to withstand any preparation or application procedures that could affect the integrity of the support.

Polymers of natural origin are favored for enzyme immobilization due to their good biocompatibility profile especially for in vivo applications. Alginate, a seaweed extract composed of chains of alternating glucuronic acid (G) and mannuronic acid (M) is by far the most widely used and tested natural polymer used in enzyme immobilization and cell microencapsulation. Alginate supports are usually made by cross-linking the anionic carboxyl group of the glucuronic acid on the polymer chain with a solution of a cationic cross-linker solution (e.g., calcium chloride, barium chloride or poly(L-lysine)). Since the G portion is the cation binding site, the ratio of G:M determines the degree of cross-linking possible for a given alginate preparation and, thus, the mechanical strength of the resulting product.

Alginate supports made by cross-linking with a calcium cation are very popular in immobilized systems. They are very easy to formulate using mild reactions, and the resulting supports have good mechanical strength. However, the Ca²⁺-alginate supports are unstable in physiological solutions containing calcium-chelating agents such as citrates, phosphate or EDTA. The chelating agents have a higher affinity for calcium ions than alginate and can extract the ions over time, leaving the support more permeable and with lower mechanical strength (Matthew et al., 1993).

In order to overcome the calcium extraction problem, other cations have been employed. Poly(L-lysine) (PLL), a cationic polymer has been used with promising results. PLL is not extracted by the regular calcium chelators. However, alginate-PLL supports have shown signs of toxicity and of eliciting immune responses when tested in in vivo models (Matthew et al., 1993). In addition, alginate-PLL supports demonstrate low mechanical strength when compared to synthetic polymer supports (Chang et al., 1999). Finally, alginate-PLL supports generally are not favored because PLL is an expensive cross-linking agent. Barium chloride, another cross-linker that is not extracted by physiological solutions, has been used to provide the barium cation to cross-link alginate. It has been reported that Ba²⁺-alginate exhibits strong mechanical strength compared to alginate-PLL and is free from the toxic effects of PLL (Zimmermann et al., 2000). Free Ba²⁺ ions, however, would inhibit K⁺ access to immobilized enzyme. Therefore, the formed Ba²⁺-alginate needs to be thoroughly washed.

Chitosan, another polymer of interest, has been used in many therapeutic and non-therapeutic areas. Chitosan is a product of the N-deacetylation of chitin, a polymer of N-acetyl-2-amino-2-deoxy-D-gluco-pyranose, which is found in shells of crab, lobster and shrimps. In addition to being safe and biocompatible, chitosan is abundant and relatively inexpensive, since its precursor is the second most abundant polysaccharide after cellulose. One disadvantage of chitosan is its limited solubility in solutions near physiological pH as the D-glucosamine residue has a pKₐ value of 6.5. Thus, chitosan is usually soluble in solutions with low pH values, such as dilute hydrochloric acid or acetic acid. This property of chitosan limits its employment in enzyme immobilization as many enzymes are not stable and can degrade at such low pH values. To overcome this limitation, the positively charged chitosan polymer has been complexed with alginate (which has a negative charge) to form an ionic network. Such a network has been found to have very high stability and good mechanical strength in physiological solutions (Gaserod et al., 1999). Antibodies, bovine serum albumin and horseradish peroxidase have all been successfully immobilized in alginate-chitosan supports. (Albarghouthi et al., 2000; Polk et al., 1994; Patel et al., 2000). Complexing with a combination of different anionic and cationic polymers has been used for the immobilization of hepatocytes (Matthew et al., 1993). Among the tested complexes, a polymeric support composed of carboxymethylcellulose (CMC)/chitosan/chondroitin sulfate-A provided good cellular growth, sufficient permeability and good mechanical strength in comparison to alginate-PLL supports when used for cell or enzyme immobilization. Other natural polymers used in immobilization systems include DEAE-dextran, poly(L-lysine), polyurethane, poly(vinyl alcohol), poly(ethylene oxide), Dextran sulfate, chondroitin sulfate A, sodium-carboxymethyl cellulose and polyacrylate.

Several synthetic polymers have also been used successfully in enzyme and cell immobilization. They offer flexibility to the user to select the desired polymer and to match it to the immobilization system intended. Synthetic polymers are not affected by microbial contamination and yield strong supports in comparison to most natural polymers. However, natural polymers are considered the first choice for use because of their safety profile and biocompatibility. Examples of synthetic polymer systems include a procedure for encapsulating cells in a photopolymerizable poly(ethylene glycol) matrix (Lesney, M., 2001). Several other polymers have been used such as acrylic acid derivatives, polymethacrylates (Eudragit®), poly(vinyl alcohol), and polyurethanes and semi-synthetic polymers such as DEAE-dextran, and carboxymethylcellulose (Taylor et al., 1991; Gemeiner, P., 1992).

### BRIEF SUMMARY OF THE INVENTION

The invention is directed to an immobilized catalytic system, and to methods of making such systems, comprising a carrier layer containing a catalytic entity and a permeable screening layer for providing controlled access between the immobilizing catalytic entity and the surrounding environment. The carrier layer includes the catalytic entity mixed with a neutral or anionic carrier polymer, which may or may not be cross-linked with a cross-linking agent. The screening layer over the carrier layer includes a matrix of a cationic polymer that is permeable to molecules processed by, produced by or acted upon by the catalytic entity but is not permeable to the catalytic entity itself. Any counter ion to the neutral or anionic carrier polymer cannot be the same as the cationic polymer of the screening layer, and any counter ion to the cationic polymer cannot be the same as the neutral or anionic carrier polymer.

The catalytic system of the invention can be prepared in many useful forms, such as round discs, thin films, microfibers, microcapsules or nanocapsules and, preferably, microspheres or nanospheres. The immobilized catalytic entity can be, for example, a protein such as an enzyme, an antibody, a ribonucleic acid, an RNA aptamer, a metal catalytic system (such as a platinum catalyst) or other chemical entity, a cellular component, a whole cell, a body tissue or a microorganism.

Examples of the neutral or anionic polymer of the carrier layer include neutral or anionic polysaccharides, polyvinyl derivatives, polymethacrylates, polyalkylene oxidizer glycols, anionic polyalkylene oxides or glycols, polycarboxylic acids, anionic surfactants, anionic phospholipids, carboxyalkylcelluloses and mixtures thereof. Preferably, the neutral or anionic carrier polymer is alginate, hyaluronate, chondroitin sulfate, poly(vinyl alcohol), poly(hydroxypropyl methacrylate), carboxymethylcellulose, acid-modified polyethylene glycol, acid-modified polyethylene oxide, heparin, dextran sulfate, methoxypoly(ethylene glycol) sulfonate or a mixture thereof. Polymers preferred for the screening layer include chitosan and other water-soluble chitin derivatives, cationic cellulose derivatives, cationic polyacrylates and mixtures thereof.

The matrix of the screening layer can be formed from the cationic polymer by crosslinking with either a covalent or an ionic crosslinking agent. Examples of covalent crosslinking agents include dialdehydes, dicarboxylic acids and salts thereof, diisocyanates, epichlorohydrin and benzoquinone, while examples of ionic crosslinking agents include salts containing divalent anions and salts containing trivalent anions. Preferred divalent or trivalent anions of ionic crosslinking agents are the sulfates, phosphates, citrates, or tripolyphosphates.

In a particularly preferred embodiment, the catalytic system of the invention is in the form of microcapsules and the microencapsulated catalytic system includes a central core comprising the catalytic entity, which is preferably an enzyme or other cellular component, mixed with a neutral or anionic carrier polymer and an outer shell surrounding the core and comprising a matrix of a cationic polymer. Depending on the cation used to crosslink the neutral or anionic carrier polymer, the central core of the system may be liquid or solid.

Theoretically, any enzyme or other catalytic entity can be immobilized according to the invention, giving a wide range of potential applications, e.g., in industrial, medical, pharmaceutical, agricultural, cosmetic and toxicological fields. Enzyme immobilization increases storage life in comparison to free enzyme. It provides a protective medium for the enzyme from the effects of stirring and mixing solutions by protecting the enzyme from the shear stress of agitation. In addition, separation of the enzyme from the reaction medium is easily achieved and does not require any chemical or physical processes. The immobilized system, for in vivo uses, requires that the carrier and screening polymers used be biocompatible and non-toxic. In addition, the system should have sufficient mechanical strength to protect the biological entity from mechanical stress. The final immobilized system should be perm-selective, allowing small molecular weight compounds to diffuse in and out, while preventing high molecular weight compounds from entering. This will offer the advantage of preventing body immune rejection in case a foreign enzyme is administered in a therapeutic treatment.

Enzyme immobilization can be used, e.g., to study drug metabolism by encapsulating drug-metabolizing enzymes and studying the reactions in vitro. This would provide a safe way to gain knowledge of toxicity and metabolism pathways that can play a role in enhancing research in the field of drug metabolism studies. The immobilized systems according to the invention can be used to activate drugs in vivo by immobilizing enzymes that can process pro-drugs. Such systems will be of great value for treating cancer and other diseases while decreasing side effects. Multiple industrial applications, such as the enzymatic production of medicinal compounds or chemicals, are contemplated. Further medical use in the field of enzyme replacement therapy is possible if an absent enzyme is immobilized and implanted in the patient. In addition, the immobilization of certain detoxifying enzymes can be used to clean the environment from harmful pesticides or to treat poisoning by chemical compounds. Either native or genetically modified cells, when immobilized according to the invention can act as delivery systems for secreted proteins. The system of the invention can also be extended to growing plant cells for making drugs.

### BRIEF DESCRIPTION OF THE DRAWINGS

Other features and advantages of the invention will be apparent from the following description of the preferred embodiments thereof and from the claims, taken in conjunction with the accompanying drawings, in which:

FIG. 1 shows, in cartoon form, an immobilized enzyme system according to the prior art;

FIG. 2 shows various possible geometric configurations of catalytic systems according to the invention;

FIG. 3 is an SEM image of a cross-section of a freeze-dried plain chitosan microcapsule;

FIG. 4A is an SEM image of a cross-section of a chitosan Ca²⁺-alginate hybrid microcapsule according to the invention;

FIG. 4B is an SEM image of a cross-section of a chitosan Ba²⁺-alginate hybrid microcapsule according to the invention;

FIG. 5 is a bar graph showing the enzyme loading percentage of a system according to the invention with different initial amounts of HRP;

FIG. 6 is a micrograph of HRP-loaded hybrid microcapsules according to the invention incubated with Amplex Red®. The dark center indicates localization of the enzyme in the core of the microcapsule;

FIG. 7 is a plot of ONP absorption versus time in the presence different ONPG concentrations for chitosan Ca²⁺-alginate hybrid microcapsules according to the invention;

FIG. 8 is a plot of ONP absorption versus time in the presence different ONPG concentrations for chitosan Ba²⁺-alginate hybrid microcapsules according to the invention;

FIG. 9 is a graph showing the stability profiles of free enzyme, enzyme loaded chitosan Ca²⁺-alginate hybrid microspheres according to the invention and enzyme loaded chitosan Ba²⁺-alginate hybrid microspheres according to the invention at 4° C.;

FIG. 10 is a graph showing the stability profiles of free enzyme, chitosan Ca²⁺-alginate hybrid microspheres with enzyme and chitosan Ba²⁺-alginate hybrid microspheres with enzyme at 25° C.;

FIG. 11 is a graph showing the stability profiles of free enzyme, chitosan Ca²⁺-alginate hybrid microspheres with enzyme and chitosan Ba²⁺-alginate hybrid microspheres with enzyme at 37° C.; and

FIG. 12 is a graph showing the stability profiles after 24 hours for the three enzyme forms at 4, 25, and 37° C.

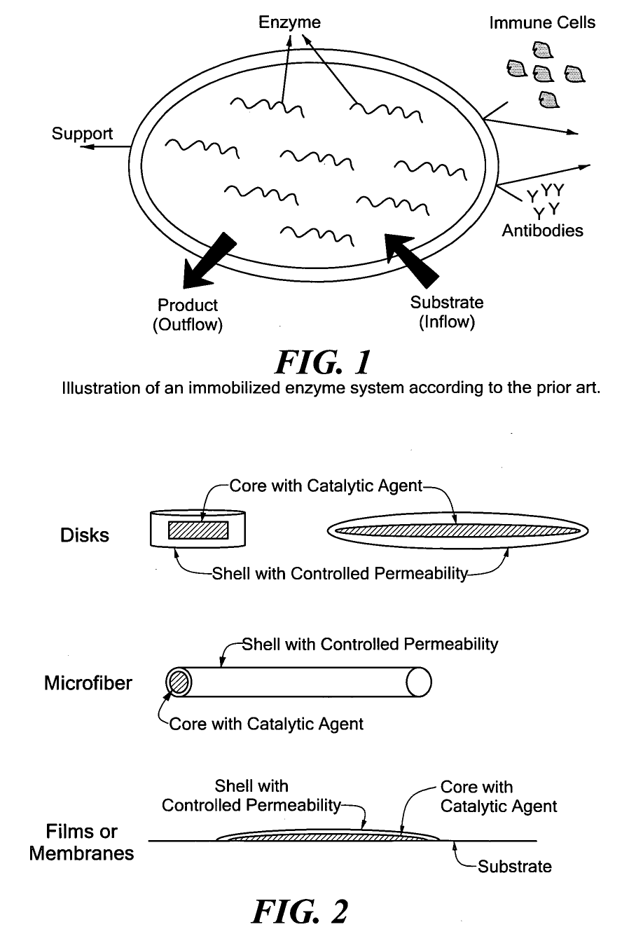

### DESCRIPTION OF THE PREFERRED EMBODIMENTS

Alginate-chitosan core-shell microcapsules according to the invention have been prepared as novel biocompatible matrix systems for enzyme immobilization, where the catalyst is confined to the core and the transport properties of the substrate and product are dictated by the permeability of the shell. Alginate as the primary core component provides several advantages. If Ca²⁺ or Ba²⁺ ions are used for crosslinking alginate, microcapsules with liquid or solid cores, respectively, can be prepared. With β-galactosidase as a model enzyme, the system of the invention achieved 60% loading efficiency with a Ca²⁺-alginate liquid core and 100% loading efficiency with a Ba²⁺-alginate solid core. The enzymatic activity of β-galactosidase in the immobilized system was determined using ONPG as a substrate. The Vₘₐₓ values for the Ca²⁺-alginate- and Ba²⁺-alginate-chitosan core-shell microcapsules were significantly lower than that of the free enzyme due to the additional layer necessary for the influx of the substrate and outflux of the product. The solid core microcapsules, prepared with Ba²⁺-alginate, however, did improve the stability of the enzyme at 37° C. as compared to the liquid core Ca²⁺-alginate microcapsules and the free enzyme.

Chitosan was selected as a material for the microcapsule shell for several reasons. First, as described earlier, it is an abundant cationic biopolymer with intra- and intermolecular hydrogen bonding ability. Various geometries such as spheres, capsules, membranes and fibers can easily be formed from chitosan (Hirano et al., 1987; Hann et al., 2002). Catalytic systems according to the invention configured in a variety of geometries are shown in FIG. 2. Second, we have developed a unique approach to modifying the surface of chitosan by complexation-interpenetration of anions (e.g., heparin, dextran sulfate, and poly(ethylene glycol)-sulfonate) to improve biocompatibility (Lesney, 2001; Sun et al., 1984; Hann et al., 2002). Surface-modified chitosan can resist protein adsorption and cell adhesion in the biological milieu. Lastly, we have also developed the technology to create chitosan membranes with controlled pore size and density, such that molecules of specific sizes can permeate through (Amiji, 1999).

The unique approach according to the invention for preparing microcapsules for the immobilization of enzymes, cells, and microorganisms permits the biological agent to be protected in the inner, biocompatible core while the outer shell is fabricated to provide a selectively permeable layer. Some potential applications of proteins and cells immobilized according to the invention are given in Table 1.

TABLE 1
Summary of some potential applications
of immobilized proteins and cells.

| Enzymes | Cells |
|---|---|
| Drug Metabolism Studies | Artificial Organ |
| Biosensors | Drug Metabolism Studies |
| Treatment of Enzyme Deficiency | Industrial Production of |
| Disorders | Alcohol |
| Detoxification of Pollutants | Production of Cytostatic |
| | Drugs |
| Synthesis of Drugs | Delivery of Therapeutic |
| | Proteins |

The following examples are presented to illustrate the advantages of the present invention and to assist one of ordinary skill in making and using the same. These examples are not intended in any way otherwise to limit the scope of the disclosure.

### Example I

### Formation of Alginate-Chitosan Hybrid Microcapsules

Chitosan microcapsules without the alginate core were formed to optimize the conditions for the final alginate-chitosan hybrid microcapsules. The chitosan solution was prepared by dissolving 0.75 gram of chitosan in 100 ml of 0.1-M acetic acid. The solution was mixed for 8 hours, then filtered through glass wool and degassed overnight. The suitable concentration of chitosan was found to be 0.75% (w/v), and the optimum cross-linking time was found to be 1.5 hours.

Tests to optimize the conditions were done for alginate bead preparation. The type of alginate (Protanal®, Pronova, Wash.) used was determined by cross-linking alginate with different G:M ratios, Protanal® LF 20/200 (55:45 G:M ratio) was found to have the strongest walls at 0.34 M CaCl₂ and 45 minutes cross-linking time. The optimum CaCl₂ concentration to cross-link the alginate was determined by cross-linking alginate in different CaCl₂ solutions (1-10% w/v). The 0.34-M solution provided strong beads. No additional strength was seen at higher CaCl₂ concentrations. To determine the cross-linking time, beads were cross-linked in a 0.34-M aqueous CaCl₂ solution for different times from 5 minutes up to 60 minutes. All beads showed good strength, and beads cross-linked for 45 minutes showed maximum strength. Further cross-linking time provided no significant improvements in strength. However, later results suggested that a cross-linking time of 5 minutes simultaneously provides sufficient strength and an improved enzyme loading percentage.

A 2.0% w/v solution of Na-alginate was prepared by dissolving 2 grams of Na-alginate (Protanal LF 20/200, Pronova, Wash.) in 100 ml of distilled water. The solution was mixed for 8 hours until the powder was completely dissolved. The solution was dropped into a 0.34-M aqueous CaCl₂ solution using a syringe with a 27½-gauge needle. The formed microspheres were allowed to sit in the CaCl₂ solution for 5 minutes. The final beads were collected, washed once with deionized distilled water, and were stored at 4° C. Alginate beads cross-linked with BaCl₂ were prepared using a 0.34 M BaCl₂ as a cross-linking solution; the cross-linking time was kept 5 minutes.

The alginate microspheres, prepared as mentioned above, were dispersed in a 0.75% (w/v) chitosan and allowed to sit for several seconds. Using a plastic dropper with the end cut to provide the appropriately sized opening, the suspended alginate beads were sucked into the dropper and then dropped into a 3% (w/v) sodium tripolyphosphate (Na-TPP) aqueous solution. The microcapsules formed were allowed to sit in the Na-TPP solution for 1.5 hours to ensure complete cross-linking. After the Ca²⁺-alginate hybrid microcapsules were formed, the core was found to have a liquid consistency due to the extraction of the Ca⁺² ions by the phosphate ions in the cross-linking step. The hybrid microcapsules containing Ba²⁺-alginate beads did not liquefy, as Ba²⁺ ions are not leached out by the phosphate ions. Thus, Ba²⁺-alginate hybrid microcapsules have a solid core.

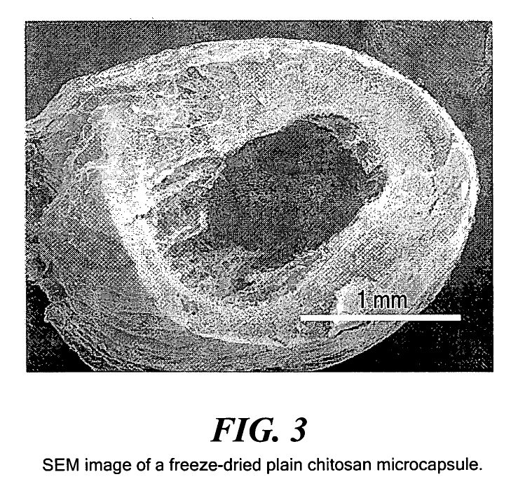

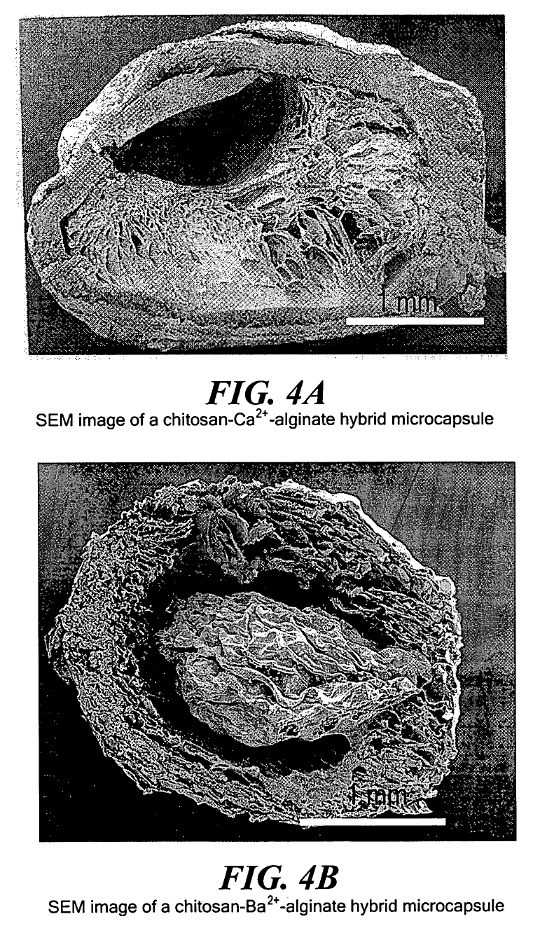

Scanning Electron Microscopy (SEM) was performed on freeze-dried control chitosan microcapsules and freeze-dried alginate-chitosan hybrid microcapsules. The surface as well as cross-sections of the capsules were analyzed (see FIGS. 3, 4A and 4B). The SEM analysis was done using magnification of 40× and 75× for the chitosan microcapsules and the alginate-chitosan hybrid microcapsules, respectively.

The strength of the microcapsules was studied by measuring the equilibrium water uptake by the hydrogels. Equilibrium water uptake of the capsules is an indicator of the mechanical strength of the capsule. When the capsule takes up water, the wall swells and the matrix becomes less compacted. As the uptake of water increases, the strength of the capsules is usually decreased. Five freeze-dried microcapsules (alginate encapsulated in chitosan) were weighed and then suspended in distilled water. After one hour, the capsules were taken out and the surface water was removed by placing the capsules on a dry Kim-wipe® tissue paper. After the excess surface water had been removed, the capsules were weighed again. Five freeze-dried capsules made up of chitosan only were tested the same way, as controls. The procedure was carried out in triplicate for both the Ca²⁺- and Ba²⁺-alginate-encapsulated chitosan and the empty chitosan microcapsules.

The equilibrium water uptake (EWU) was calculated using the following equation (Anderson et al., 2001):

EWU=[(Wₛ−W_{d})/Wₛ]×100%

where Wₛ is the weight of the swollen capsules and W_{d} is the weight of the dry capsules.

The mechanical strength of the calcium and barium hybrid microcapsules as well as plain chitosan microcapsules was tested, and the force required to rupture the microcapsules was measured. In the case of dry microcapsules, chitosan (control) microcapsules had the lowest mechanical strength, having an average burst force of 16.1 g. Liquid core microcapsules (Ca²⁺-alginate core) required a force of 132 g to burst. Solid core microcapsules had 2 burst points: the first representing the breaking of the outer chitosan shell happened at 14 g, and the second, corresponding to the Ba²⁺-alginate bead breaking, was at 707 g. The wet chitosan microcapsules had a burst point of 9.6 g. The wet Ca²⁺-alginate hybrid microcapsules required as force of 3 g. The wet Ba²⁺-alginate hybrid microcapsules required a force of 21.1 g to break. Not being bound by any theory, it is believed that the reason the wet Ca²⁺-alginate hybrid microcapsules were weaker than the chitosan control microcapsules is that the control microcapsules have a smaller size (represented by a smaller diameter −2.133 mm for chitosan and 2.453 mm for the Ca²⁺-alginate hybrid microcapsules). In addition, the Ca²⁺-alginate hybrid microcapsules have a higher inner osmotic pressure due to the alginate present in the core, which is absent in the control microcapsules. Ba²⁺-alginate hybrid microcapsules had a higher mechanical strength in both the dry and the wet state.

### Example II

### Determination of the Molecular Weight Cut-Off Point of Cross-Linked Chitosan

The permeability of chitosan is the rate-limiting step in the microencapsulated system. In order to estimate the molecular weight cut-off point of the cross-linked chitosan, a model representing the chitosan layer was developed in which a thin membrane acts as a layer surrounding the alginate core. The permeability coefficients for vitamin B2 (molecular weight 376 daltons) and vitamin B12 (molecular weight 1355 daltons) representing low-molecular weight substrates, and myoglobin (molecular weight 14,000 daltons) representing a high molecular weight substrate, were studied.

Chitosan solution was prepared by dissolving the polymer (750 kDa, 87.6% deacetylation) obtained from Pronova Biopolymers (Raymond, Wash.) in 0.1-M acetic acid to make a 0.75% (w/v) solution. Films were made by pouring 10 ml of the solution into a Petri dish (100×15 mm) and air-drying for up to 48 hours. The resulting films were dipped into a 3.0% (w/v) aqueous sodium tri-polyphosphate (Na-TPP, Sigma Chemical Company, St. Louis, Mo.) solution and kept for 1.5 hours. The cross-linked membranes were washed with distilled water once and stored in PBS at 4° C.

The thickness of the membranes was determined using a caliper after cross-linking the membranes and washing them with distilled water. The mean wet-thickness of the membranes was found to be 50±4.0 μm.

SEM analysis of freeze-dried chitosan membranes cross-linked with Na-TPP was performed. SEM analysis was performed with an AMR-1000 scanning electron microscope (Amray Instruments, Bedford, Mass.) at a voltage of 10 kV. The membrane surface and cross-section images were scanned at magnifications of 13,000×.

Using a side-by-side diffusion apparatus, the permeability coefficients of vitamin B2, vitamin B12, and myoglobin were determined. Briefly, each apparatus was composed of a donor compartment and a receptor compartment, with a 15-ml capacity. The donor and receptor compartments were separated by the membrane.

The donor compartment was filled with 15 ml of vitamin B2, vitamin B12, or myoglobin solution, and the receptor compartment was filled with 15 ml of phosphate buffered-saline (PBS) pH 7.4. The concentrations of vitamin B2, vitamin B12 and myoglobin were 0.1, 1.0, and 0.1 mg/ml respectively. The receptor compartment was stirred and temperature was controlled in both compartments at 37° C. by a circulating water bath. Samples were taken from the receptor compartment periodically for up to 24 hours. The absorbance values of vitamin B2 at 440 nm, vitamin B12 at 361 nm, and myoglobin at 410 nm were measured using a Shimadzu 160U UV/IV spectrophotometer (Columbia, Md.). The concentration of the diffused compound into the receptor compartment was calculated from calibration curves constructed earlier. The permeability coefficient P was calculated from the following equation:

In(Cₒ/Cₜ)=(PSt)/(hV)

where Cₒ is the initial concentration of each compound in the donor compartment. Cₜ is the concentration at a given time. S is the surface area of the membrane (1.77 cm²). V is the volume in the donor compartment and h is the thickness of the membrane. The plot of in (Cₒ/Cₜ) against t/h was used to calculate P.

The permeability coefficients of vitamin B2, vitamin B12 and myoglobin through cross-linked chitosan at 37° C. are presented in Table 2.

TABLE 2
Permeability coefficients of different solutes
through cross-linked chitosan membranes.

| | | Permeability |
|---|---|---|
| | Molecular Weight | Coefficient (cm²/min × |
| Compound | (daltons) | 10⁶) |
| Vitamin B2 | 376 | 31.9 ± 0.37 |
| Vitamin B12 | 1355 | 7.46 ± 1.47 |
| Myoglobin | 14,000 | 0.34 ± 0.89 |

As can be seen the membrane had a higher permeability coefficient for vitamin B2 than vitamin B12 due to the relative low molecular weight of vitamin B2. Myoglobin permeated at only a low level. Using the log(permeability coefficient) versus log (molecular weight) profile, we calculated the molecular cut-off of the membrane to be 20,000 daltons and the preferred pore size to be <100 nm. Based on this study, it was determined that a chitosan layer would provide a selectively permeable screen for substrate and product diffusion in the enzyme-immobilized system of the invention.

### Example III

### Immobilization of Horseradish Peroxidase to Determine Loading Efficiency and Retained Bioactivity

Horseradish peroxidase (HRP) is an enzyme that catalyzes the conversion of hydrogen peroxide to water. O-phenylenediamine OPD(H₂)— a chromogen for HRP catalyzed reactions—is converted from a colorless compound to a yellow product OPD(—H₂) when HRP catalyzes the conversion of hydrogen peroxide to water. The yellow product can be detected using visible spectroscopy at 495 nm. Briefly, an OPD(H₂) solution (0.25 mg/ml) was prepared in a 0.1 M citrate buffer at pH 6. Then 30% (v/v) hydrogen peroxide was added to provide a final concentration of 0.6% (v/v). One hundred microliters of hydrogen peroxide solution was placed in each well of a 96-wells microtiter plate. One hundred microliters of HRP (molecular weight 40,000, ICN) solution was then added to the wells, and after 10 minutes, the reaction was stopped by the addition of 50 μl of 1.0-N sodium hydroxide. HRP was tested for the efficiency of enzyme loading into the alginate beads. HRP was used in a concentration of 50 U/ml. The loading of the enzyme was measured by detecting enzymatic activity and comparing it to a standard curve of HRP activity.

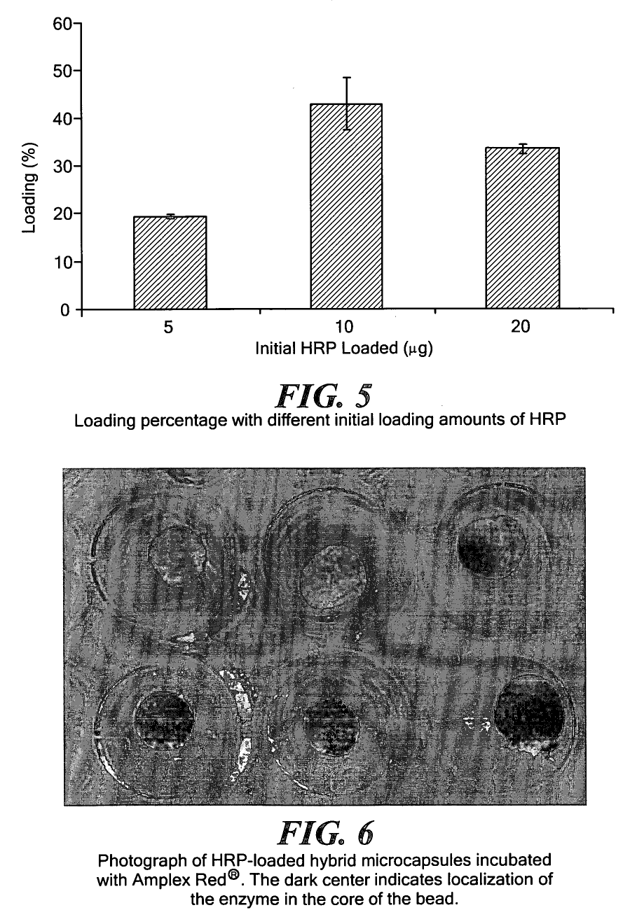

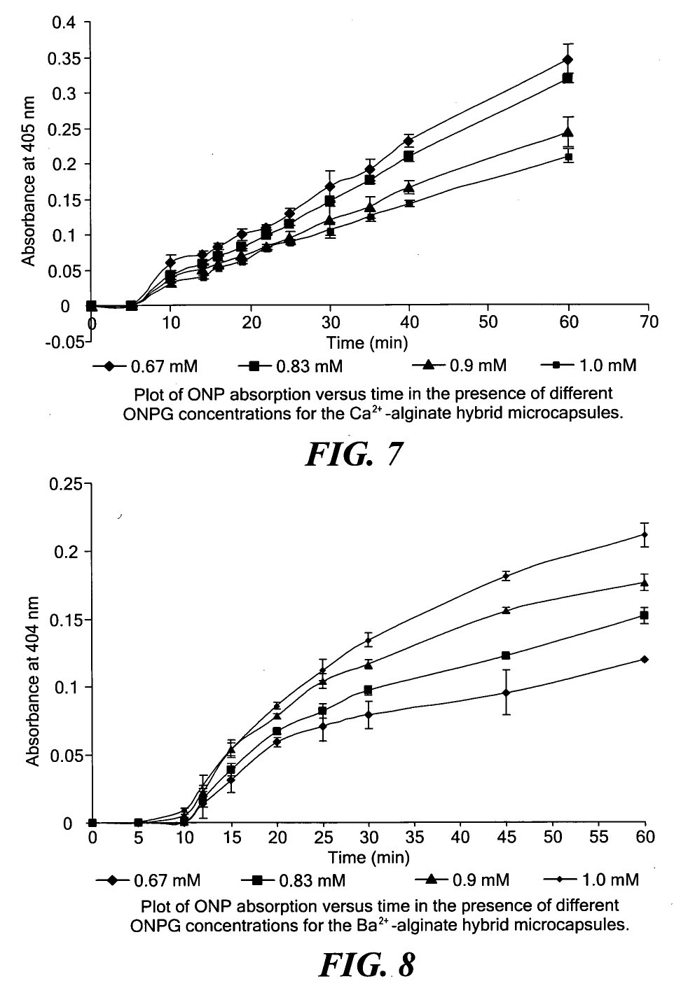

The HRP enzyme was suspended in Ca²⁺-alginate solution and the beads were made as described earlier. The beads were cross-linked for 45 minutes. HRP was added to the 1 ml of alginate solution to give a final concentration of 5, 10 and 20 μg/ml. FIG. 5 shows the results of loading with various concentrations. A loading concentration of 10 μg/ml appeared to give the best results.

A group of beads was loaded with 10 μg/ml HRP, or 100 ng of enzyme per bead, as described above, and the effect of cross-linking time on loading efficiency was studied. The beads cross-linked for 5 minutes were able to entrap approximately 100% of the enzyme (compared to a standard solution), while the beads cross-linked for 45 minutes entrapped only 42.8% of HRP. The loading efficiency appears to have increased with a shorter cross-linking time because of a decreased contact time of the beads with the cross-linking solution. Thus, leaching of the enzyme out of the beads was decreased. Therefore, in subsequent studies, the cross-linking time was decreased from 45 to 5 minutes to increase the loading efficiency, although the strength of the alginate bead was slightly reduced.

A fluorescent method was used to measure HRP kinetics. Amplex Reds (Molecular Probes, Eugene, Oreg.), a fluorophore for HRP, is converted to the highly fluorescent resorufin giving a red color with an excitation/emission wavelengths of 570/582 nm. A 200 μM Amplex Reds solution was prepared using 50 μM phosphate buffer pH 7.4. Hydrogen peroxide was used at a concentration of 20 mM. HRP was used at a concentration of 10 μg/100 μl, the amount of immobilized HRP that is equivalent to 100% loading. The final volume was brought to 3.0 ml with phosphate buffer. Due to the instability of Amplex Red® in the presence of light or when in solution for a short time, the kinetics of the enzyme were not measured. An enzyme having a more stable substrate and product was chosen for further studies. However, beads loaded with HRP were incubated with the Amplex Red for 30 minutes in a phosphate buffer pH 7.4 to qualitatively detect enzymatic activity. FIG. 6 shows a qualitative image of Ca²⁺-alginate hybrid microcapsules loaded with HRP and incubated with the substrate Amplex Red

### Example IV

### Immobilization of β-galactosidase and Use as a Model Enzyme

Loading of β-galactosidase was studied by incorporating the enzyme into alginate beads and then measuring the enzymatic activity of β-galactosidase after dissolving the beads in 3% (w/v) Na-TPP aqueous solution. β-galactosidase converts o-nitrophenolgalactopyranoside (ONPG) to o-nitrophenol (ONP) and galactose. ONP has a yellow color and can be detected using visible spectroscopy at 405 nm.

β-galactosidase (333 U/ml) was mixed in 3 ml of 2% (w/v) alginate solution at room temperature for 3 minutes. Following the mixing process, 1 ml (100 beads) of the alginate solution was dropped using a 3.0 ml syringe with a 27½ gauge needle into either a 0.34 M CaCl₂ aqueous solution or a 0.34 M BaCl₂ aqueous solution. The beads were cross-linked for 5 minutes, removed from the cross-linking solution and then washed with distilled water.

The Ca²⁺-alginate loaded beads were placed in 20 ml of 3% Na-TPP solution and stirred until the beads completely dissolved. As a control, alginate beads without the enzyme were formed and dissolved in 20 ml of 3% w/v Na-TPP. After complete dissolution, the control solution was spiked with 100 μl of 333 U/ml β-galactosidase (theoretical amount of enzyme present in the 100 beads of alginate). One hundred microliters of the dissolved bead solution was added to 2.9 ml of 1 mM ONPG and allowed to react for 5 minutes. In addition, 100 μl of the spiked control solution was added to 2.9 ml of 1 mM ONPG. The reaction was stopped with 100 μl of 1.0M sodium carbonate. The concentration of enzyme in the control solution was considered 100%, and the concentration of the enzyme in the sample solution was compared to the control solution. Loading of the enzyme in the Ca²⁺-alginate beads resulted in a 60% yield, while retention of the enzyme in the Ba²⁺-alginate beads was 100%. The binding of the alginate polymer chains in the presence of the Ba²⁺ ion are stronger, leading to the high enzyme retention within the matrix of the polymer. The binding in the presence of the Ca²⁺ ions is possibly slower and the binding is not as strong, which allows the enzyme to leach out into the cross-linking solution.

The enzyme concentration in the BaCl₂ beads was measured indirectly by measuring the enzyme concentration in the cross-linking solution. Two methods were used; UV absorbance at 280 nm to determine protein concentration and enzymatic activity. Ba²⁺-alginate beads cannot be dissolved in Na-TPP solution. It is important to note that when enzyme loading in Ca²⁺-alginate beads was determined from the cross-linking solution, the results were similar to the method described above. The kinetics of β-galactosidase activity was studied in the free enzyme (non-immobilized) and the immobilized enzyme form. The rational was to see if the outer shell membrane (chitosan) had any effect on the kinetics of the enzyme. When the alginate core is liquefied (Ca²⁺-alginate beads), the rate-limiting step for enzymatic catalysis is assumed to be diffusion across the outer chitosan membrane. In the case of the non-liquified core (Ba²⁺-alginate beads), diffusion is presumed to be hampered by both the outer chitosan layer and the cross-linked alginate matrix.

Briefly, the kinetics of the free enzyme was studied by conventional enzyme kinetics assessments. The enzyme concentration was held constant while the substrate concentration was varied. The initial rate was calculated for each concentration. For the free enzyme kinetics studies, a final volume in each tube was 3.0 ml. An enzyme concentration of 3.33 U/100 μl was used. The amounts of ONPG used ranged from 0.05 to 0.25 μmole. The reactions were started and absorption readings at 405 nm were determined. Readings were taken initially at 5 second intervals, and then after 20 seconds, the absorption readings were taken at 10 second intervals.

The kinetics of the immobilized enzyme were studied in a similar fashion as the free enzyme with some modifications to accommodate for the diffusion of substrate and product. Briefly, 8 enzyme-containing beads (either Ca²⁺--alginate or Ba²⁺-alginate hybrids) were placed in each vial containing 2, 2.5, 2.75 and 3 ml of 1 mM ONPG. Phosphate buffer (0.1 M, pH 7.4) was added to any vial to complete the volume up to 3 ml. Samples were taken at time intervals starting at 5 minutes and for up to 60 minutes and absorbance was measured at 405 nm to determine the appearance of product. The following effectiveness ratio was used as a comparison parameter for immobilized systems. The effectiveness factor (EF) can be calculated according to the following formula (Gemeiner, P., 1992, Shuler et al., 2002, Kennedy et al., 1985):

EF=Vₘₐₓ(Immobilized enzyme)/Vₘₐₓ(Free enzyme)

The (EF) value gives an indication of the barrier effect the immobilization has on the enzyme activity. If the value of (EF) is equal to or greater than one, then there is no effect on diffusion due to the immobilization process. If the (EF) is smaller than one, then immobilization has an effect on substrate and product diffusion. The (EF) value is usually less than one in the case of physical immobilization.

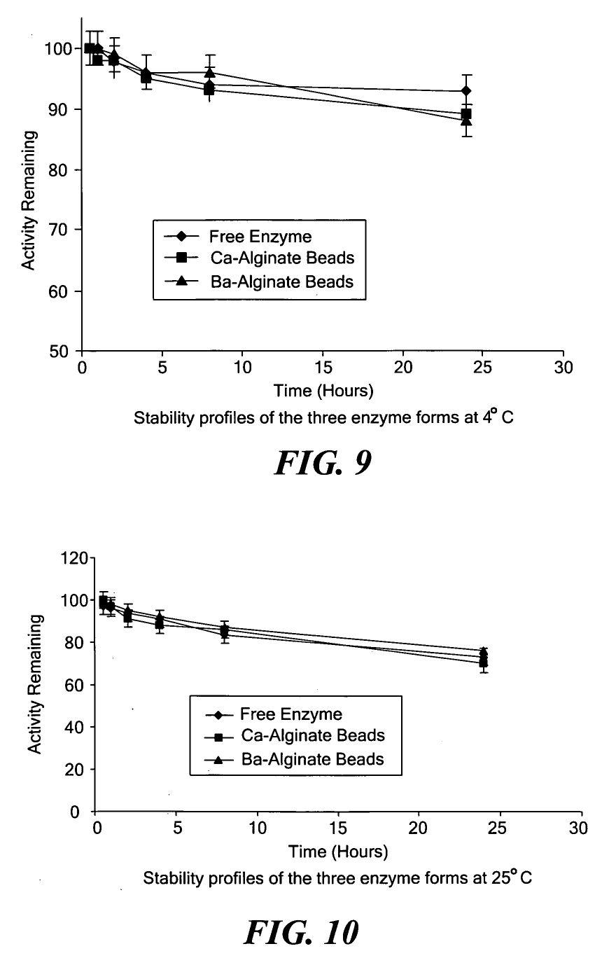

As shown in FIGS. 9 and 10, a lag time before product detection was observed. This lag time was due to the diffusion barrier for both the substrate ONPG and the product ONP by the capsule wall. In the case of Ca²⁺-alginate hybrids (FIG. 9), a lag time of around 7 minutes was observed before the product could be detected, and at 10 min. up to 60 min. an increase in the absorption was observed, indicating that the conversion of ONPG was occurring continuously. In the case of Ba²⁺-alginate hybrids (FIG. 10), a longer lag time was observed; this effect is attributed to the cross-linked Ba²⁺-alginate matrix, which did not liquefy (unlike Ca²⁺-alginate). In addition, the amount of product produced was lower over a given period of time.

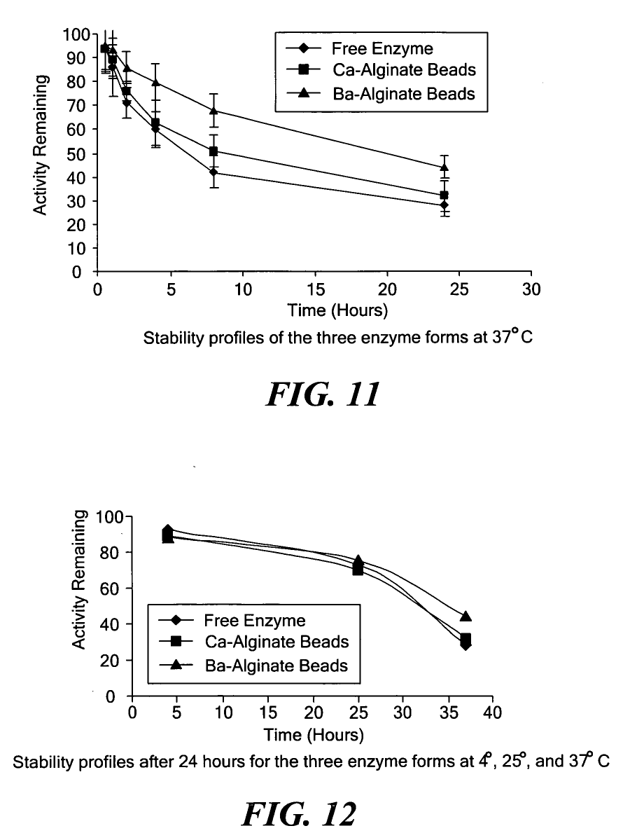

The stability of β-galactosidase was studied in both the free and the immobilized form at 4, 25, and 37° C. The free enzyme was diluted to a concentration of 20 U/ml and placed in substrate solutions at the three different temperature. Initial UV absorbance of the reaction with 1-mM substrate was measured and taken as reference. The hybrid microcapsules were loaded with an enzyme concentration of 0.5 U/bead. Referring to FIGS. 9-12, it can be seen that at 4° C., there was not much of a difference in terms of enzyme stability between the free enzyme and the immobilized enzyme forms (93% for the free enzyme, 89% for the Ca²⁺-alginate hybrids, and 88% for the Ba²⁺-alginate hybrids). The major difference in stability was observed at 37° C., where the activity remaining of the free enzyme dropped to 28% in comparison to 32% in the Ca²⁺-alginate hybrids and 44% in the Ba²⁺-alginate hybrids. The increased stability is explained by the ability of the surrounding matrix to shield the enzyme from the effects of the environment.

### REFERENCES

Aggarwal, N., H. HogenEsch, P. Guo, A. North, M. Suckow, and S. Mittal. Biodegradable alginate microspheres as a delivery system for naked DNA. Canadian Journal of Veterinary Research, 63: 148-152 (1999).
Albarghouthi, M., D. Abu Fara, M. Saleem, T. El-Taher, K. Matalka, and A. Badwan. Immobilization of antibodies on alginate-chitosan beads. International Journal of Pharmaceutics, 206: 23-34 (2000).
Amiji, M., Surface modification of chitosan to improve blood compatibility. Recent Research Development in Polymer Science, 3: 31-39 (1999).
Anderson, D., T. Nguyen, P. K Lai, and M. Amiji. Evaluation of the permeability and blood-compatibility of membranes formed by physical interpenetration of chitosan with PEO/PPO/PEO triblock copolymers. Journal of Applied Polymer Science, 80: 1274-1284 (2001).
Boppana, V., R. Lynn, and J. Ziemniak. Immobilized sulfatase: β-glucuronidase enzymes for the qualitative and quantitative analysis of drug conjugates. Journal of Pharmaceutical Sciences, 78: 127-131 (1989).
Boyd, M., L. Burka, B. Wilson, and H, Sasame. In vitro studies on the metabolic activation of the pulmonary toxin 4-ipomeanol, by rat lung and liver microsomes. Journal of Pharmacology and Experimental Therapeutics, 207: 677-686 (1978).
Chang, P., J. Van Raamsdonk, G. Hortelano, S. Barsoum, N. Macdonald, and T. Stockley. The in vivo delivery of heterologous proteins by microencapsulated recombinant cells. Trends in Biotechnology, 17: 78-83, (1999).
Daly, M., and D. Knorr. Chitosan-alginate complex coacervate capsules: effects of calcium chloride, plasticizers, and polyelectrolytes on mechanical stability. Biotechnology Progress, 4: 76-81 (1988).
De Hann, B., H. Van Goor, and P. De Vos. Processing of immunoisolated pancreatic islets: Implications for histological analyses of hydrated tissue. Biotechniques, 32: 612-619 (2002).
Drevon, G., and A. Russel. Irreversible immobilization of diisopropylphosphatase in polyurethane polymers. Biomacromolecules, 1: 571-576 (2000).
Dulik, D., and C. Fenselau. Use of immobilized enzymes in drug metabolism studies. FASEB, 2: 2235-2240 (1998).
Farghali, H., J. Martinek, L. Kamenikova, V. Bencko, and S. Hynie, Immobilized and perfused hepatocytes in drug research. Progress in Hepato-Pharmacology, 1:262-268 (1995).
Fernandez-Salguero, P., C. Gutierrez-Merino, and A. Bunch. Effect of immobilization on the activity of rat hepatic microsomal cytochrome P450 enzymes. Enzyme and Microbial Technology, 15: 100-104 (1993).
Frings, K., M. Koch, and W. Hartmeier. Kinetic resolution of 1-phenyl ethanol with high enantioselectivity with native and immobilized lipase in organic solvents. Enzyme and Microbial Technology. 25: 303-309 (1999).
Gåserφd, O., A. Sannes, and G. Skjåk-Br K. Microcapsules of alginate-chitosan. II. A study of capsule stability and permeability. Biomaterials, 20: 773-783 (1999).
Gemeiner, P., (ed). 1992. Enzyme Engineering Immobilized Biosystems. Ellis Horwood, Chichester, U K.
Gordon, G., and P. Skett 1999. Introduction to Drug Metabolism. Stanley Thornes (Publishers) Ltd, Cheltenham, U K.
Gordon, R., S. Feaster, A. Russell, K. LeJeune, D. Maxwell, D. Lenz, M. Ross and B. Doctor. Chemico-Biological Interactions, 119-120: 463-470 (1999).
Hirano, S., Y. Noishiki, J, Kinugawa, H. Higashijima, and T. Hayashi. 1987. In G. G. Gebelein (ed). Advances in Biomedical Polymers. Plenum Press New York, N.Y., p 285.
Kennedy, J. and C. White. 1985. In A. Wiseman (ed) Handbook of Enzyme Biotechnology. Ellis Horwood, Chichester, U K.
Kneser, U., P. kaufmann, H. Fiegel, J. Pollok, D. Kluth, H. Herbst, and X. Rogiers. Long-term differentiated function of heterotopically transplanted hepatocytes on three-dimensional polymer matrices. Journal of Biomedical Materials Research. 47: 494-503 (1999).

Lesney, M., The use of encapsulated living cells provides added possibilities for complex therapeutics. Modern Drug Discovery, 45-50 (2001).
Liang, J., Y. Li, and V. Yang. Biomedical application of immobilized enzymes. Journal of Pharmaceutical Sciences, 89: 979-990 (2000).
Lowry, O., N. Rosenbough, A. Farr, and R. Randall. Protein measurement with the folin phenol reagent. Journal of Biological Chemistry, 55:265-275 (1951).
Matthew, H., S. Salley, W. Peterson, and M. Klein. Complex coacervate microcapsules for mammalian cell culture and artificial organ support. Biotechnology Progress, 9: 510-519 (1993).
Patel, R., and T. Speaker. Water-based microsphere delivery system for proteins. Journal of Pharmaceutical Sciences, 89: 9-15 (2000).
Polk, A., B. Amsden, K. De Yao, T. Peng, and M. Goosen. Controlled release of albumin from chitosan-alginate microcapsules. Journal of Pharmaceutical Sciences, 83: 178-185 (1994).
Protonal LF 20/200 Product Specification. Pronova Biopolymer, Wash.
Schenkman, J., H. Remmer, and R. Estabrook, Spectral studies of drug interactions with hepatic microsomal cytochrome. Molecular Pharmacology, 3:113-123 (1967).
Shuler, M. and F. Kargi. 2002. In Bioprocess Engineering Basic Concepts 2^{nd} ed. Prentice Hall PTR, Upper Saddle River, N.J. Singh, A., A. Flounders, J. Volponi, C. Ashley, K. Wally and J. Schoeniger. Development of sensors for direct detection of organophosphates. Part 1: immobilization, characterization and stabilization of acetylcholinesterase and organophosphate hydrolase on silica supports. Biosensors and Bioelectronics, 14: 703-713 (1999).
Sun, A., G. O'Shea, and M. Goosen. Injectable microencapsulated islet cells as a bioartificial pancreas. Applied Biochemistry and Biotechnology, 10: 87-99 (1984).
Taylor, R., M. Kierstan, and M. Coughlan. 1991. In R. Taylor (ed). Protein Immobilization Fundamentals and Applications. Marcel Dekker Inc, New York, N.Y.
Trejtnar, F., L. Skalova, B. Szotakova, and V. Wsol. Use of rat hepatocytes immobilized in agarose gel threads for biosynthesis of metabolites of potential cytostatic. Experimental Toxicology and Pathology, 51: 432-435 (1999).
Wang, E., S. Overgaard, J. Scharer, N. Bols, and M. Moo-Young. Occlusion immobilization of hybridoma cells in chitosan. Biotechnology Techniques, 2: 133-136 (1988).
Yoo, I. K., G. Seong, H. Chang, and J. Park. Encapsulation of Lactobacillus casei cells in liquid-core alginate capsules for lactic acid production, Enzyme and Microbial Technology, 19: 428-433 (1996).
Zihnio\check{g}lu, F., and A. Telefoncu. Substrate specificity and the use of chitosan-entrapped rabbit hepatic microsomal UDP-glucuronyl transferase for detoxification. Artificial Cells, Blood Substitutes and Immobilization Biotechnology, 23: 533-543 (1995).
Zimmermann, U., S. Mimietz, H. Zimmermann, M. Hillgärtner, H. Schneider, J. Ludwig, C. Hasse, A, Hasse, M. Rothmund, and G. Fuhr. Hydrogel-based non-autologous cell and tissue therapy. Biotechniques, 29:564-581 (2000).

While the present invention has been described in conjunction with a preferred embodiment, one of ordinary skill, after reading the foregoing specification, will be able to effect various changes, substitutions of equivalents, and other alterations to the compositions and methods set forth herein. It is therefore intended that the protection granted by Letters Patent hereon be limited only by the definitions contained in the appended claims and equivalents thereof.

What is claimed is:

1. An immobilized catalytic system comprising
a carrier layer comprising a catalytic entity mixed with a cross-linked neutral carrier polymer or a cross-linked anionic carrier polymer and counter ion therefor; and
a screening layer over said carrier layer, wherein said screening layer comprises a cross-linked matrix of a cationic polymer and counter ion therefor, wherein said cationic polymer matrix is permeable to molecules processed by, produced by or acted upon by said catalytic entity but not permeable to said catalytic entity, and wherein said counter ion to said anionic carrier polymer is not said cationic polymer and said counter ion to said cationic polymer is not said anionic carrier polymer.

2. The catalytic system of claim 1, wherein said system is immobilized by being encapsulated and wherein, in said encapsulated system, said cross-linked carrier layer has the form of a central core and said cross-linked screening layer has the form of an outer shell surrounding said core carrier layer.

3. The catalytic system of claim 1, wherein the form of said system is selected from the group consisting of round discs, thin films, microfibers, microcapsules, nanocapsules, microspheres and nanospheres.

4. The catalytic system of claim 1, wherein said catalytic entity is selected from the group consisting of proteins, antibodies, ribonucleic acids, RNA aptamers, metal catalytic systems or other chemical entities, cellular components, whole cells, tissues and microorganisms.

5. The catalytic system of claim 1, wherein said neutral or anionic carrier polymer is selected from the group consisting of neutral or anionic polysaccharides, polyvinyl derivatives, polymethacrylates, polyalkylene oxides or glycols, anionic polyalkylene oxides or glycols, polycarboxylic acids, anionic surfactants, anionic phospholipids, carboxyalkylcelluloses and mixtures thereof.

6. The catalytic system of claim 5, wherein said neutral or anionic carrier polymer is selected from the group consisting of alginate, hyaluronate, poly(vinyl alcohol), poly(hydroxypropyl methacrylate), carboxymethylcellulose, acid-modified polyethylene glycol, acid-modified polyethylene oxide, heparin, dextran sulfate, methoxypoly(ethylene glycol) sulfonate and mixtures thereof.

7. The catalytic system of claim 1, wherein said neutral or anionic carrier polymer is cross-linked with a covalent cross-linking agent.

8. The catalytic system of claim 1, wherein said neutral or anionic carrier polymer is cross-linked with an ionic cross-linking agent.

9. The catalytic system of claim 1, wherein said cationic polymer is selected from the group consisting of chitosan and other water-soluble chitin derivatives, cationic cellulose derivatives, cationic polyacrylates and mixtures thereof.

10. The catalytic system of claim 1, wherein said cationic polymer is cross-linked with a covalent cross-linking agent.

11. The catalytic system of claim 10, wherein said covalent cross-linking agent is selected from the group consisting of dialdehydes, dicarboxylic acids and salts thereof, diisocyanates, epichlorohydrin and benzoquinone.

12. The catalytic system of claim 1, wherein said cationic polymer is cross-linked with an ionic cross-linking agent.

13. The catalytic system of claim 12, wherein said ionic cross-linking agent is selected from the group consisting of salts containing divalent anions and salts containing trivalent anions.

14. The catalytic system of claim 13, wherein said divalent or trivalent anions are sulfates, phosphates, citrates, or tripolyphosphates.

15. The catalytic system of claim 2, wherein said central core is liquid.

16. The catalytic system of claim 2, wherein said central core is solid.

17. The catalytic system of claim 2, wherein said catalytic entity in said central core is a macromolecule, an organelle or a whole cell.

18. The catalytic system of claim 17, wherein said catalytic entity is an enzyme.

19. The catalytic system of claim 17, wherein said catalytic entity is a cellular component.

20. A method of making an immobilized, microencapsulated catalytic system, said method comprising the steps of:
providing an aliquot of a catalytic entity, wherein said catalytic entity is a macromolecule, an organelle or a whole cell;
mixing said catalytic entity with a solution of a neutral carrier polymer or with an anionic carrier polymer and counter ion therefor to form a loaded carrier polymer solution;
delivering drops of said loaded carrier polymer solution into a cross-linking solution for said neutral or anionic carrier polymer, whereby microspheres of cross-linked loaded carrier polymer are formed;
isolating said microspheres of cross-linked loaded carrier polymer;
suspending said microspheres in a solution of a cationic polymer and counter ion therefor, whereby said cationic polymer solution coats said microspheres; and
delivering drops of said suspended, coated microspheres into a cross-linking solution for said cationic polymer, whereby microspheres of said immobilized, microencapsulated catalytic system are formed.

21. The method of claim 20, wherein said neutral or anionic carrier polymer is an alginate salt,
said cross-linking solution for said alginate salt is a solution of a calcium salt;
said cationic polymer is chitosan; and
said cross-linking solution for said chitosan is a solution of a phosphate salt, whereby said resulting microspheres of said immobilized, microencapsulated catalytic system comprise a liquid central core mixed with said catalytic entity and said liquid central core is surrounded by a semi-permeable outer shell.

22. The method of claim 20, wherein said neutral or anionic carrier polymer is an alginate salt,
said cross-linking solution for said alginate salt is a solution of a barium salt;
said cationic polymer is chitosan; and
said cross-linking solution for said chitosan is a solution of a phosphate salt, whereby said resulting microspheres of said immobilized, microencapsulated catalytic system comprise a solid central core mixed with said catalytic entity and said solid central core is surrounded by a semi-permeable outer shell.
